(19) United States
(12) Patent Application Publication
(10) Pub. No.: US 2005/0238674 A1
(43) Pub. Date: Oct. 27, 2005

(54) Stable injectable compositions

(75) Inventors: Lawrence John Penkler, Port Elizabeth (ZA); Barry Paul Daisley, Port Elizabeth (ZA)

Correspondence Address: NIXON PEABODY, LLP, 401 9TH STREET, NW, SUITE 900, WASHINGTON, DC, 20004-2128, US

(73) Assignee: Shimoda Biotech (Pty) Ltd., Port Elizabeth (ZA)

(21) Appl. No.: 10/999,155

(22) Filed: Nov. 30, 2004

Related U.S. Application Data
(60) Provisional application No. 60/551,351, filed on Mar. 10, 2004.

(51) Int. Cl. A61K 31/724; A61K 31/195; A61K 9/00

(52) U.S. Cl. 424/400; 514/58; 514/567

(57) ABSTRACT

This invention relates to a stable parenteral aqueous solutions comprising either (a) diclofenac or a pharmaceutically acceptable diclofenac salt and a cyclodextrin, or (b) an inclusion complex of diclofenac or a pharmaceutically acceptable diclofenac salt and a cyclodextrin, or a mixture of (a) and (b), which are suitable for intramuscular and intravenous administration. The solutions contain diclofenac or diclofenac salt, cyclodextrin, and an antioxidant selected from monothioglycerol, or a combination of ethylene diamine tetra-acetic acid and N-acetyl-cysteine.

### BACKGROUND TO THE INVENTION

Diclofenac is a leading non-steroidal anti-inflammatory drug (NSAID). The drug has been in clinical use for over two decades as a NSAID with analgesic, anti-inflammatory and anti-pyretic activity. Historically, diclofenac has been associated mainly with chronic management of inflammatory and degenerative forms of rheumatism as well as treatment of painful musculoskeletal conditions, acute attacks of gout, painful post-operative and post-traumatic inflammation and pain following dental surgery. For these conditions the drug has been available in delayed release enteric coated tablets, sustained release tablets, suppositories and ampoules for strict intramuscular injection. More recently, diclofenac has become available in rapid acting oral preparations for short term treatment of acute conditions. Since 1995, diclofenac sodium is available in the UK and Scandinavia as an intravenous infusion indicated for moderate to severe post-operative pain, or for the prophylaxis of post-operative pain.

Conventionally formulated diclofenac sodium injections are limited to intramuscular administration. This limitation has arisen, not as a consequence of the intravenous safety profile, but principally due to the physico-chemical properties of the drug, summarized as follows:
Poor aqueous solubility of the sodium salt—Diclofenac has a particularly high tendency to crystallize from aqueous and organic solutions. Physically stable solutions containing at least 25 mg/ml of diclofenac sodium necessitates the use of potent solubilizing cosolvents, such as macrogols and benzyl alcohol. These cosolvents have an unfavourable intravenous safety profile and are associated with venous sequelae, high haemolytic and sensitising potential (see Reed, K. W. et al, J. Par. Sci. Technol. 39(2) (1985) 64-68).
Susceptibility to oxidation—Diclofenac's tendency to oxidize in solution necessitates formulation with antioxidants, for example sulphite salts. In the commercial European intramuscular product, antioxidants such as sodium metabisulphite or sodium disulphite are usually used. Sulphite salts have been implicated in serious hypersensitivity reactions causing, for example, broncho-constriction (see Gunnison, A. F. et al, CRC Critical Reviews in Toxicology 17(3)(1987) 185-214).
pH and Osmolality—The high pH of the marketed product (ca. 8.5) required to render diclofenac sodium soluble and the hyperosmolar nature of the formulation contribute to the discomfort which is frequently experienced at the site of the injection when administered intramuscularly.
Injection Volume—Owing to poor solubility, the commercial product is formulated as 25 mg diclofenac sodium per millilitre. The recommended dosage is 75 mg and therefore the product is given as a 3 millilitre intramuscular injection. This is above the recommended volume of 2 millilitres for intramuscular injection accepted by the United States Food and Drug Administration.

U.S. Pat. No. 5,679,660 to Farmarc Nederland BV teaches a method of preparing an injectable pharmaceutical or veterinary composition which comprises either diclofenac or a salt thereof and 2-hydroxypropyl beta-cyclodextrin with a preferred concentration of diclofenac of 25 mg per millilitre. This reference discloses a method whereby the aqueous solubility of diclofenac was increased with the aid of a cyclodextrin to the extent that it could be formulated into a parenteral formulation containing 75 mg diclofenac per 3 ml. The formulatory volume of 3 ml is not problematic with regard to the intravenous dosage route, as the drug will possibly be given by infusion, but as far as the intramuscular dosage form is concerned, a volume of 3 ml will not meet with FDA approval.

It has been found that solutions of diclofenac sodium in 2-hydroxypropyl beta-cyclodextrin prepared according to U.S. Pat. No. 5,679,660 with a diclofenac sodium concentration of 25 mg per millilitre are stable for up to 12 months at room temperature and at least 24 months under refrigerated conditions. After 12 months at room temperature and 4 months at elevated temperature (e.g. 40° C.), appearance of visible insoluble particulate matter occurs which progresses with time. In order to satisfy a 24 month pharmaceutical shelf-life, the injectable product should be stored under refrigerated conditions.

A refrigerated parenteral product however has the disadvantage of discomfort upon injection due to the low temperature of the injected product coupled with the increased cost of product storage.

It is an object of this invention to provide a parenteral dosage form of diclofenac which addresses the aforementioned limitations and which may be used for both intramuscular and intravenous administration.

### SUMMARY OF INVENTION

According to the invention there is provided a stable parenteral aqueous solution comprising either (a) diclofenac or a pharmaceutically acceptable diclofenac salt and a cyclodextrin, or (b) an inclusion complex of diclofenac or a pharmaceutically acceptable diclofenac salt and a cyclodextrin, or a mixture of (a) and (b), which is preferably suitable for intramuscular and intravenous administration, the solution containing:
diclofenac or diclofenac salt, preferably diclofenac sodium;
cyclodextrin, typically 2-hydroxypropyl beta-cyclodextrin; and
an antioxidant selected from monothioglycerol, or a combination of ethylene diamine tetra-acetic acid and N-acetyl-cysteine.

The molar ratio of diclofenac to 2-hydroxypropyl beta-cyclodextrin is preferably 1:1.5 to 1:2.5, most preferably 1:2.

Typically, the solution comprises 20 mg to 45 mg, preferably more than 25 mg, most preferably 37.5 mg, diclofenac or diclofenac salt per millilitre solution.

Where the antioxidant is monothioglycerol, the monothioglycerol may comprise 0.1 to 10 mg, preferably 0.1 to 5 mg, most preferably 5 mg, per millilitre solution.

Where the antioxidant is a combination of ethylene diamine tetra-acetic acid and N-acetyl-cysteine, the ethylene diamine tetra-acetic acid may comprise 0.05 to 1 mg, preferably 0.5 mg, per millilitre solution and the N-acetyl-cysteine may comprise 0.1 to 2 mg, preferably 1 mg, per millilitre solution.

Advantageously, the solution is in the form of a unit dose that does not exceed 2 millilitres.

### DETAILED DESCRIPTION OF THE INVENTION

The inventor has found a way to prepare an aqueous solution comprising either (a) diclofenac or a pharmaceutically acceptable diclofenac salt and a cyclodextrin, or (b) an inclusion complex of diclofenac or a pharmaceutically acceptable diclofenac salt and a cyclodextrin, or a mixture of (a) and (b), which is not only capable of having a concentration of diclofenac or diclofenac salt of more than 25 mg per millilitre of solution, but is also stable and does not need to be refrigerated when packed in clear glass pre-fillable syringes. By “stable” is meant that the solution can be stored for at least 12 months at room temperature and at least 6 months at elevated temperature (40° C.) without the appearance of particulate matter which is visible to the eye.

The use of an antioxidant in the form of monothioglycerol (MTG) or a combination of ethylene diamine tetra-acetic acid (EDTA) and N-acetyl-cysteine (NAC) has been found to not only increase the diclofenac solubility to the extent that it is possible to dissolve 75 mg of diclofenac-cyclodextrin into a final volume of 2 ml (which means that the solubility of diclofenac (which is a very poorly water soluble drug) has been increased to such an extent that it could be formulated in a final volume 33% less than that proposed in U.S. Pat. No. 5,679,660), but also effectively stabilises the solution preventing the formation of particulate matter at elevated temperature in pre-fillable syringes, ampoules and vials.

The solution may be formulated in unit dose form, each unit dose containing from 10 mg to 150 mg diclofenac or diclofenac salt inclusive, more preferably from 25 mg to 75 mg inclusive, most preferably 75 mg, in a volume not exceeding 2 millilitres.

The 2-hydroxypropyl beta-cyclodextrin (HPBCD) is selected from derivatives with a degree of substitution of between 2.5 and 10 hydroxypropyl substitutents per beta-cyclodextrin molecule, more preferably between 3.5 and 8 hydroxypropyl substitutents per beta-cyclodextrin molecule. The molar ratio of diclofenac to 2-hydroxypropyl beta-cyclodextrin is 1:1 to 1:10, more preferably 1:1.5 to 1:2.5, most preferably 1:2.

The injectable stabilised solution of the invention may be prepared by methods known in the art (e.g. U.S. Pat. No. 5,679,660, the content of which is incorporated herein by reference).

The stabilised injectable solution of the invention may be packed into suitable containers known in the art (for example glass ampoules, vials, cartridges, pre-filled syringes and the like). The glass should preferably be clear glass.

The stabilised injectable solution of the invention may be intravenously administered by admixture with non dextrose infusion fluids.

The stabilized injectable solution of the invention is suitable for intravenous and intramuscular use, saving money with regard to manufacturing cost and provides the patient with less discomfort due to a smaller intramuscular injection volume.

The stabilized injectable solution of the invention need not be stored under refrigerated conditions to provide a shelf life of at least 24 months, saving refrigeration costs during transport and storage, and alleviating patient discomfort during administration.

The antioxidants of the invention show advantages over a control solution containing no antioxidant and solutions containing other antioxidants, namely NAC or EDTA by themselves, sodium formaldehyde sulphoxilate (SFS) by itself and a combination of SFS and EDTA. Tables 1 and 2 below show stability evaluations of 75 mg per 2 ml diclofenac sodium formulations prepared according to the process disclosed in U.S. Pat. No. 5,679,660 stored at 40° C. for 3 and 6 months respectively. It is evident from Tables 1 and 2 that whereas the NAC EDTA combination and monothioglycerol formulations according to the invention are stable after 6 months at 40° C., formulations containing NAC or EDTA by themselves, SFS by itself and a combination of SFS and EDTA are not stable.

TABLE 1
Stability Evaluation Of 75 mg/2 ml Diclofenac Sodium
Antioxidant Batches at 40° C. For 3 Months

| | Chemical | pH trend | Appearance/Particulate | |
|---|---|---|---|---|
| Antioxidant | stability | at 40° C. | matter | Comments |
| Control (No | Acceptable | Upward | Discolouration; physical | Not stable |
| additives) | level of | | instability (+++) | (continued to 6 |
| | known | | | months as |
| | degradant | | | control) |
| EDTA 0.05% | Acceptable | Slightly | Discolouration; physical | Product not |
| | level of | upward | instability (+++) | stabilised |
| | known | | | sufficiently-trial |
| | degradant | | | discontinued |
| NAC 0.1% | Acceptable | Slightly | Stable | Complies, trial |
| | level of | upward | | continued |
| | known | | | |
| | degradant | | | |
| NAC 0.1% + EDTA | Acceptable | Stable | Stable | Complies, trial |
| 0.05% | level of | | | continued |
| | known | | | |
| | degradant | | | |
| Monothioglycerol | Acceptable | Slightly | Stable | Complies, trial |
| 0.1% | level of | upward | | continued |
| | known | | | |
| | degradant | | | |
| Monothioglycerol | Acceptable | Slightly | Stable | Complies, trial |
| 0.5% | level of | upward | | continued |
| | known | | | |
| | degradant | | | |
| Thioglycerol 0.1% + | Acceptable | Slightly | Stable | Complies, trial |
| EDTA 0.05% | level of | upward | | continued |
| | known | | | |
| | degradant | | | |
| SFS 0.005% | New | Upward | Physically unstable (+++) | Not stable-trial |
| | degradant | | | discontinued |
| | which | | | |
| | exceeds | | | |
| | 0.1% m/m | | | |
| SFS 0.1% | New | Slightly | Discolouration; physically | Not stable-trial |
| | degradant | upward | unstable, but more stable | discontinued |
| | which | | than 0.005% solution (++) | |
| | exceeds | | | |
| | 0.1% m/m | | | |
| SFS 0.005% + EDTA | Acceptable | Slightly | Physically unstable (++) | Not stable-trial |
| 0.05% | level of | upward | | discontinued |
| | known | | | |
| | degradant | | | |

Key:
(+) = Very few colloidal particulates, fibres or filling artefacts
(++) = Evidence of physical instability under light
(+++) = Physical instability readily observable with the naked eye

TABLE 2
Stability Evaluation of 75 mg/2 ml Diclofenac Sodium
AntiOxidant Batches at 40° C. after 6 Months

| | Chemical | pH trend | Appearance/Particulate | |
|---|---|---|---|---|
| Antioxidant | stability | at 40° C. | matter | Comments |
| Control (No | Acceptable | Upward | Discolouration; physical | Not stable |
| additives) | level of | | instability (+++) | (Control) |
| | known | | | |
| | degradant | | | |
| NAC 0.1% | Acceptable | Slightly | Discolouration; physical | Not stable |
| | level of | upward | instability (+++) | |
| | known | | | |
| | degradant | | | |
| NAC 0.1% + | Acceptable | Slightly | Clear, straw colour - | Formulation |
| EDTA 0.05% | level of | upward | some particulate matter | possible |
| | known | | | |
| | degradant | | | |
| Monothioglycerol | Acceptable | Slightly | Clear, straw colour - | Formulation |
| 0.1% | level of | upward | some particulate matter (+) | possible |
| | known | | | |
| | degradant | | | |
| Monothioglycerol | Acceptable | Slightly | Clear, straw colour - | Formulation |
| 0.5% | level of | upward | some particulate matter (+) | possible |
| | known | | | |
| | degradant | | | |
| Monothioglycerol | Acceptable | Slightly | Clear, straw colour - | Formulation |
| 0.1% + EDTA | level of | upward | some particulate matter (+) | possible |
| 0.05% | known | | | |
| | degradant | | | |

Key:
(+) = Very few colloidal particulates, fibres or filling artefacts
(++) = Evidence of physical instability under light
(+++) = Physical instability readily observable with the naked eye

The invention will now be described in more detail with reference to the following non-limiting examples.

### EXAMPLE 1

The unit composition of a first preferred formulation of the invention is provided in Table 3 below:

TABLE 3

| Ingredient | Quantity/2 ml |
|---|---|
| Diclofenac Sodium | 75 mg |
| Hydroxypropyl-β-cyclodextrin | 666 mg |
| N-acetyl-L-cysteine | 2 mg |
| Disodium edetate (EDTA) | 1 mg |
| Water for Injection to | 2 ml |
| Final pH | 6.5-8.5 |

### EXAMPLE 2

The unit composition of a second preferred formulation of the invention is provided in Table 4 below:

TABLE 4

| Ingredient | Quantity/2 ml |
|---|---|
| Diclofenac Sodium | 75 mg |
| 2-Hydroxypropyl-β-cyclodextrin | 666 mg |
| Monothioglycerol | 10 mg |
| Water for Injection to | 2 ml |
| Final pH | 6.5-8.5 |

### EXAMPLE 3

Laboratory-scale formulations given in Examples 1 and 2 of the present invention were manufactured according to Example 4 of U.S. Pat. No. 5,679,660 and filled into clear glass prefillable syringes and placed on a stability program. Table 5 below summarizes the results obtained:

TABLE 5
STABILITY DATA FOR STABILIZED 75 mg/2 ml DICLOFENAC
SODIUM-HPB SOLUTIONS

| | |
|---|---|
| Stabilised by: 0.5% m/v | Stabilised by 0.05% m/v |
| monothioglycerol | EDTA & 0.1% m/v NAC |

T = 6 months

| | 25° C. | 40° C. | | 25° C. | 40° C. |
|---|---|---|---|---|---|
| Appearance | Clear | Clear, straw | Appearance | Clear, | Clear, straw |
| | colourless | coloured | | colourless | coloured |
| | solution | solution | | solution | solution |
| PH | 7.44 | 8.12 | PH | 7.17 | 8.16 |
| Particulate | Free from | Free from | Particulate | Free from | Free from |
| Matter | visible | visible | Matter | visible | visible |
| | particulate | particulate | | particulate | particulate |
| | matter | matter | | matter | matter |
| Assay (HPLC) | ˜100% | ˜99% | Assay (HPLC) | ˜100% | ˜99% |
| (% of T = 0) | | | (% of T = 0) | | |
| Indolinone | <0.1% m/m | 0.57% | Indolinone | <0.1% | 0.53% m/m |
| Other | None | None | Other | None | None |
| degradants | detected | detected | degradants | detected | detected |

Control solutions at 40° C. for 6 months showed heavy precipitation of an insoluble red-coloured material.

After 24 months at 25° C. the solution containing monothioglycerol remained clear and slightly coloured, free from visible particulate matter. The associated solution containing N-acteyl-cysteine/EDTA was clear but more darkly colored than the monothioglycerol solution.

### EXAMPLE 4

To produce 250 75 mg/2 ml diclofenac sodium units for IM or IV injection, 500 ml water for injection (WFI) is purged with nitrogen gas to reduce the oxygen content to less than 0.5 mg/l. The water was heated to 50° C. Processing continues under a nitrogen gas blanket. 166.675 g of HPBCD (DS 4.69) is added to 60% of the WFI batch volume and is mixed until dissolved. The solution is then allowed to cool to room temperature. The solution is pre-filtered with a 0.45 μg filter, followed by the addition of 2.5 g MTG. The solution is stirred until all the MTG is dissolved. The pH is then adjusted to 4.5. 18.75 g diclofenac sodium is added to the solution and stirred until dissolved and made up to 100% volume with WFI and the pH is adjusted to 7.4, should it be required. The resultant 75 mg/2 ml diclofenac sodium solution is sterilized by filtration with 0.22 μm filters and filled into pre-sterilized ampoules/vials under aseptic conditions. The ampoules/vials are sealed aseptically under nitrogen. The formulation contains 75.0±3.75 mg/2 ml diclofenac sodium, as determined by validated HPLC.

### EXAMPLE 5

To produce 250 75 mg/2 ml diclofenac sodium units for IM or IV injection, 500 ml water for injection (WFI) is purged with nitrogen gas to reduce the oxygen content to less than 0.5 mg/l. The water was heated to 50° C. Processing continues under a nitrogen gas blanket. 166.675 g of HPBCD (DS 4.69) is added to 60% of the WFI batch volume and is mixed until dissolved. The solution is then allowed to cool to room temperature. The solution is pre-filtered with a 0.45 μg filter, followed by the addition of 0.5 g NAC and 0.25 g EDTA. The solution is stirred until all the NAC and EDTA is dissolved. The pH is then adjusted to 4.5. 18.75 g diclofenac sodium is added to the solution and stirred until dissolved and made up to 100% volume with WFI and the pH is adjusted to 7.4, should it be required. The resultant 75 mg/2 ml diclofenac sodium solution is sterilized by filtration with 0.22 μm filters and filled into pre-sterilized ampoules/vials under aseptic conditions. The ampoules/vials are sealed aseptically under nitrogen. The formulation contains 75.0±3.75 mg/2 ml diclofenac sodium, as determined by validated HPLC.

### EXAMPLE 6

A production trial batch was produced according to the method as described in Example 5, whereby 15000 units of 75 mg/2 ml diclofenac sodium IM or IV units were produced. The stability of the formulations was monitored for 12 months at 25° C. and 6 months at 40° C.

The stability trial results are summarized in Table 6 below:

TABLE 6
SCREENING STABILITY TRIAL DATA

| Diclofenac Sodium 75 mg/2 ml (control) | | | Diclofenac Sodium 75 mg/2 ml (NAC/EDTA) | | |
|---|---|---|---|---|---|
| Stoichiometry ˜1:2 | | | Stoichiometry ˜1:2 | | |
| Control Batch (no stabilisers) | | | Control Batch (0.05% EDTA + 0.1% NAC) | | |
| Amber ampoule | | | Amber ampoule | | |
| CONTROL BATCH | | | STABILISED BATCH | | |
| | 25° C. | 40° C. | | 25° C. | 40° C. |

T = 3 months

| | | | | | |
|---|---|---|---|---|---|
| Appearance | Clear | Clear | Appearance | Clear | Clear, |
| | colourless | yellow | | colourless | slightly |
| | solution | solution | | solution | straw |
| | | | | | colourled |
| | | | | | solution |
| pH | 7.25 | 7.74 | pH | 7.14 | 7.56 |
| Particulate | Present | Present | Particulate | Free from | Free from |
| Matter | (under | | Matter | visible | visible |
| | direct light | | | particulate | particulate |
| | beam) | | | matter | matter |
| Assay | 73.95 mg/2 ml | 73.57 mg/2 ml | Assay | 72.96 mg/2 ml | 72.90 mg/2 ml |
| | (99.8% of T = 0) | (99.3% of T = 0) | | (99.9% of T = 0) | (99.8% of T = 0) |
| Indolinone | <0.1% | 0.23% m/m | Indolinone | nd | 0.31% m/m |
| Other | nd | | Other | nd | nd |

T = 6 months

| | | | | | |
|---|---|---|---|---|---|
| Appearance | Straw | Hazy, | Appearance | Clear | Clear, |
| | coloured | yellow — | | colourless | slightly |
| | solution | orange | | solution | straw |
| | | solution | | | colourled |
| | | | | | solution |
| pH | 7.43 | 8.13 | pH | 7.25 | 8.47 |
| Particulate | Present | Present | Particulate | Free from | Free from |
| Matter | | | Matter | visible | visible |
| | | | | particulate | particulate |
| | | | | matter | matter |
| Assay | 74.81 mg/2 ml | 74.28 mg/2 ml | Assay | 73.47 mg/2 ml | 72.88 mg/2 ml |
| | (100.9%) | (100.2%) | | (100.6%) | (99.8%) |
| Indolinone | <0.1% | 0.42% m/m | Indolinone | <0.1% | 0.62% m/m |
| Other | nd | 0.11% m/m | Other | nd | nd |

T = 9 months

| | | | | | |
|---|---|---|---|---|---|
| Appearance | Straw | N/A | Appearance | Clear | N/A |
| | coloured | | | colourless | |
| | solution | | | solution | |
| pH | 7.53 | N/A | pH | 7.36 | N/A |
| Particulate | Present | N/A | Particulate | Free from | N/A |
| Matter | | | Matter | visible | |
| | | | | particulate | |
| | | | | matter | |
| Assay | 74.18 mg/2 ml | N/A | Assay | 73.32 mg/2 ml | N/A |
| | (100.1%) | | | (100.4%) | |
| Indolinone | <0.1% | N/A | Indolinone | 0.1% | N/A |
| Other | nd | N/A | Other | nd | N/A |

T = 12 months

| | | | | | |
|---|---|---|---|---|---|
| Appearance | Straw | N/A | Appearance | Clear | N/A |
| | coloured | | | colourless | |
| | solution | | | solution | |
| pH | 7.62 | N/A | pH | 7.28 | N/A |
| Particulate | Present | N/A | Particulate | Free from | N/A |
| Matter | | | Matter | visible | |
| | | | | particulate | |
| | | | | matter | |
| Assay | 75.01 mg/2 ml | N/A | Assay | 74.59 mg/2 ml | N/A |
| | (101.2%) | | | (102.1%) | |
| Indolinone | <0.1% m/m | N/A | Indolinone | 0.1% m/m | N/A |
| Other | nd | N/A | Other | nd | N/A |

No physical or chemical instability was observed for the trial batches after 12 months at 25° C. and 6 months at 40° C. There was no apparent difference between the upright or vertical orientation of the formulations.

1. A stable parenteral aqueous solution comprising either (a) diclofenac or a pharmaceutically acceptable diclofenac salt and a cyclodextrin, or (b) an inclusion complex of diclofenac or a pharmaceutically acceptable diclofenac salt and a cyclodextrin, or a mixture of (a) and (b), the solution containing:
diclofenac or diclofenac salt;
cyclodextrin; and
an antioxidant selected from monothioglycerol, or a combination of ethylene diamine tetra-acetic acid and N-acetyl-cysteine.

2. The stable parenteral aqueous solution according to claim 1, wherein the diclofenac salt is diclofenac sodium.

3. The stable parenteral aqueous solution according to claim 1, wherein the cyclodextrin is 2-hydroxypropylbeta-cyclodextrin.

4. The stable parenteral aqueous solution according to claim 3, wherein the molar ratio of diclofenac to 2-hydroxypropyl beta-cyclodextrin is 1:1.5 to 1:2.5.

5. The stable parenteral aqueous solution according to claim 4, wherein the molar ratio of diclofenac to 2-hydroxypropyl beta-cyclodextrin is 1:2.

6. The stable parenteral aqueous solution according to claim 1, comprising more than 25 mg diclofenac or diclofenac salt per millilitre solution.

7. The stable parenteral aqueous solution according to claim 6, comprising 37.5 mg diclofenac or diclofenac salt per millilitre solution.

8. The stable parenteral aqueous solution according to claim 1, wherein the antioxidant is monothioglycerol and the monothioglycerol comprises 0.1 to 10 mg per millilitre solution.

9. The stable parenteral aqueous solution according to claim 8, wherein the monothioglycerol comprises 0.1 to 5 mg per millilitre solution.

10. The stable parenteral aqueous solution according to claim 9, wherein the monothioglycerol comprises 5 mg per millilitre solution.

11. The stable parenteral aqueous solution according to claim 1, wherein the antioxidant is a combination of ethylene diamine tetra-acetic acid and N-acetyl-cysteine, and the ethylene diamine tetra-acetic acid comprises 0.05 to 1 mg per millilitre solution and the N-acetyl-cysteine comprises 0.1 to 2 mg per millilitre solution.

12. The stable parenteral aqueous solution according to claim 11, wherein the ethylene diamine tetra-acetic acid comprises 0.5 mg per millilitre solution and the N-acetyl-cysteine comprises 1 mg per millilitre solution.

13. The stable parenteral aqueous solution according to claim 1, in the form of a unit dose that does not exceed 2 millilitres.
